(19) United States
(12) Patent Application Publication
(10) Pub. No.: US 2007/0221218 A1
(43) Pub. Date: Sep. 27, 2007

(54) Dry Powder Drug Containment System Packages with Tabs, Inhalers and Associated Methods

(76) Inventors: Jeffrey Alan Warden, Raleigh, NC (US); John Denny, Cary, NC (US); Michaek King, Durham, NC (US); Patrick D. Lopath, Stamford, CT (US)

Correspondence Address: MYERS BIGEL SIBLEY & SAJOVEC, PO BOX 37428, RALEIGH, NC, 27627, US

(21) Appl. No.: 10/595,466

(22) PCT Filed: Oct. 26, 2004
(86) PCT No.: PCT/US04/35424

§ 371 Date: Nov. 27, 2006

Related U.S. Application Data
(60) Provisional application No. 60/514,733, filed on Oct. 27, 2003.
(60) Provisional application No. 60/605,484, filed on Aug. 30, 2004.

(51) Int. Cl. A61M 15/00; A61B 19/00

(52) U.S. Cl. 128/203.15; 604/403

(57) ABSTRACT

Dry powder drug containment packages with tab members and meted dose sealed drug compartments can be used in an inhaler having a flow chamber with a hook member that opens the drug compartments and/or a portion that flexes to vibrate the dry powder to facilitate fluidic active dispersion for inhalation.

### RELATED APPLICATIONS

This application claims priority to U.S. Provisional Application Ser. No. 60/514,733 filed Oct. 27, 2003 and U.S. Provisional Application Serial No. 60/605,484 filed Aug. 30, 2004, the contents of the above applications are hereby incorporated by reference as if recited in full herein.

### FIELD OF THE INVENTION

The present invention relates to drug containment and/or dispensing systems suitable for dry powders formulated for delivery as inhalant aerosols.

### BACKGROUND OF THE INVENTION

Dry powder inhalers (DPIs) represent a promising alternative to pressurized pMDI (pressurized meted dose inhaler) devices for delivering drug aerosols without using CFC propellants. See generally, Crowder et al., 2001: an Odyssey in Inhaler Formulation and Design, Pharmaceutical Technology, pp. 99-113, July 2001; and Peart et al., New Developments in Dry Powder Inhaler Technology, American Pharmaceutical Review, Vol. 4, n. 3, pp. 37-45 (2001). Typically, the DPIs are configured to deliver a powdered drug or drug mixture that includes an excipient and/or other ingredients. Conventionally, many DPIs have operated passively, relying on the inspiratory effort of the patient to dispense the drug provided by the powder. Unfortunately, this passive operation can lead to poor dosing uniformity because inspiratory capabilities can vary from patient to patient (and sometimes even use-to-use by the same patient, particularly if the patient is undergoing an asthmatic attack or respiratory-type ailment which tends to close the airway).

Generally described, known single and multiple dose dry powder DPI devices use: (a) individual pre-measured doses, such as capsules containing the drug, which can be inserted into the device prior to dispensing; or (b) bulk powder reservoirs which are configured to administer successive quantities of the drug to the patient via a dispensing chamber which dispenses the proper dose. See generally Prime et al., Review of Dry Powder Inhalers, 26 Adv. Drug Delivery Rev., pp. 51-58 (1997); and Hickey et al., A new millennium for inhaler technology, 21 Pharm. Tech., n. 6, pp. 116-125 (1997).

In operation, DPI devices strive to administer a uniform aerosol dispersion amount in a desired physical form (such as a particulate size) of the dry powder into a patient's airway and direct it to a desired deposit site(s). If the patient is unable to provide sufficient respiratory effort, the extent of drug penetration, especially to the lower portion of the airway, may be impeded. This may result in premature deposit of the powder in the patient's mouth or throat.

A number of obstacles can undesirably impact the performance of the DPI.

For example, the small size of the inhalable particles in the dry powder drug mixture can subject them to forces of agglomeration and/or cohesion (i.e., certain types of dry powders are susceptible to agglomeration, which is typically caused by particles of the drug adhering together), which can result in poor flow and non-uniform dispersion. In addition, as noted above, many dry powder formulations employ larger excipient particles to promote flow properties of the drug. However, separation of the drug from the excipient, as well as the presence of agglomeration, can require additional inspiratory effort, which, again, can impact the stable dispersion of the powder within the air stream of the patient. Unstable dispersions may inhibit the drug from reaching its preferred deposit/destination site and can prematurely deposit undue amounts of the drug elsewhere.

Further, many dry powder inhalers can retain a significant amount of the drug within the device, which can be especially problematic over time. In addition, the hygroscopic nature of many of these dry powder drugs may also require that the device be cleansed (and dried) at periodic intervals.

Some inhalation devices have attempted to resolve problems attendant with conventional passive inhalers. For example, U.S. Pat. No. 5,655,523 proposes a dry powder inhalation device which has a deagglomeration/aerosolization plunger rod or biased hammer and solenoid, and U.S. Pat. No. 3,948,264 proposes the use of a battery-powered solenoid buzzer to vibrate the capsule to effectuate the release of the powder contained therein. These devices propose to facilitate the release of the dry powder by the use of energy input independent of patient respiratory effort. U.S. Pat. No. 6,029,663 to Eisele et al. proposes a dry powder inhaler delivery system with a rotatable carrier disk having a blister shell sealed by a shear layer that uses an actuator that tears away the shear layer to release the powder drug contents. The device also proposes a hanging mouthpiece cover that is attached to a bottom portion of the inhaler. U.S. Pat. No. 5,533,502 to Piper proposes a powder inhaler using patient inspiratory efforts for generating a respirable aerosol and also includes a rotatable cartridge holding the depressed wells or blisters defining the medicament holding receptacles. A spring-loaded carriage compresses the blister against conduits with sharp edges that puncture the blister to release the medication that is then entrained in air drawn in from the air inlet conduit so that aerosolized medication is emitted from the aerosol outlet conduit. The contents of these patents are hereby incorporated by reference as if stated in full herein.

More recently, Hickey et al., in U.S. patent application Ser. No. 10/434,009 and PCT Patent Publication No. WO 01/68169A1 and related U.S. National Stage patent application Ser. No. 10/204,609, have proposed a DPI system to actively facilitate the dispersion and release of dry powder drug formulations during inhalation using piezoelectric polymer film elements which may promote or increase the quantity of fine particle fraction particles dispersed or emitted from the device over conventional DPI systems. The contents of these documents are hereby incorporated by reference as if recited in full herein.

Notwithstanding the above, there remains a need for alternative inhalers and/or blister packages that can be used with dry powder inhalers.

### Summary of Embodiments of the Invention

Embodiments of the present invention include multi-dose drug containment system packages adapted for use in an inhaler. The packages include: (a) a support member comprising a plurality of spaced apart drug compartments, each drug compartment having a sealant material detachably sealed thereto; and (b) a plurality of spaced apart tab members, a respective tab member attached to a forward and/or rearward portion of a respective drug compartment sealant material. A respective tab member is operatively associated with at least one drug compartment so that, in operation, the respective tab member pulls the associated sealant material away from at least one drug compartment to release a drug held therein.

In particular embodiments, the drug package may include meted amounts and/or doses of dry powder disposed in each drug compartment. The doses may be of the same drug or different drugs. The tab members may be configured as generally downwardly extending loop members disposed proximate an outermost edge portion of the support member.

Other embodiments are directed to dry powder inhalers. The inhalers include: (a) an elongate chamber having opposing first and second end portions, a floor, and a ceiling with dry powder entry window, the first end portion merging into an inhaler mouth port and the second end portion merging into an air inlet port such that in fluid communication; (b) a vibrator operatively associated with a portion the elongate chamber; and (c) a multi-dose dry powder package comprising a plurality of spaced apart discrete meted amounts of particulate dry powder in respective sealed drug compartments. In operation, at least one of the compartments is configured to align with the chamber entry window to release at least one meted amount of dry powder therein.

In some embodiments, the vibrator may be configured to contact the elongate chamber floor and/or the inhaler port and the air inlet port may be generally axially aligned about opposing end portions of the inhaler and configured to be oriented about a generally horizontal plane during inhalation. The dry powder inhaler has a length from a forward edge portion to a rearward edge portion thereof when held in an operative position and the elongate chamber may have a length that extends across at least a major portion of the length of the inhaler.

In particular embodiments, the dry powder package may have a generally structurally rigid unitary primary body with cavities defining a ceiling and sidewalls of respective drug compartments and the floors may include a flexible material and be configured to extend across the respective cavities to define a generally planar sealant layer.

The inhaler may include a display configured to display the number of doses available in the inhaler and a controller in communication with the display. The controller may be configured to communicate with the disposable multi-dose dry powder package held therein, to automatically determine the number of doses available for dispensing on the disposable dry powder package and automatically count down and display the number available.

In some embodiments, the drug compartments comprise a detachable sealant material and the inhaler can include a translating hook member. In operation, the hook member is operatively associated with one or more drug compartments as the dry powder package rotates the drug compartments into an active dispensing position in the inhaler. The hook member can be configured to engage and peel the sealant material off the one or more drug compartments held in the active dispensing location and release the dry powder held therein into the window of the elongate channel.

Yet other embodiments are directed to multi-dose dry powder inhalers. The inhalers include: (a) an inhaler having a housing body with a mouthpiece port and a spaced apart air inlet port disposed upstream thereof; (b) a multi-dose dry powder package comprising a plurality of spaced apart dry powder drug compartments held in the inhaler; and (c) a hook member disposed in the inhaler to translate between forward and rearward positions to engage a target tab to selectively pull the associated floor sealant material off at least one drug compartment during operation.

In some embodiments, the dry powder package can include: (a) a unitary dry powder package body comprising a plurality of spaced apart drug compartments, the body having opposing top and bottom primary surfaces with a plurality of spaced apart wells having a depth formed therein, a respective well defining at least a portions of a respective drug compartment; a meted amount of dry powder held in the drug compartments; (b) a detachable floor sealant material extending across each drug compartment and sealably attached to the bottom primary surface of the dry powder package body to capture the dry powder in a respective drug compartment; and (c) a plurality of spaced apart generally downwardly extending tabs attached to an outermost edge portion of a portion of the floor sealant material.

In some embodiments, the unitary body is a generally rigid elastomeric material and is configured with a generally closed upper surface that defines a ceiling and at least a portion of a sidewall of a respective drug compartment. In some particular embodiments, the drug compartments can be configured as pairs of drug compartments with each pair of drug compartments holding a different meted dry powder drug therein. A common sealant material segment can define the floor of each pair of compartments. The common sealant material segment can be operatively associated with a single tab. In operation, the hook member engages a single tab and the common sealant material is pulled off the pair of compartments to release both drugs therein for generally concurrent release into the inhaler to thereby allow combination inhalation dry powder drug delivery in operative position in an inhaler.

Still other embodiments are directed to methods of operating an inhaler. The methods include: (a) moving at least one drug compartment held on a dry powder package into a dispensing position above a dry powder entry window in an inhaler, the dry powder package having a plurality of sealed drug compartments, each having a meted amount of dry powder held captured therein above a releaseably attached floor sealant material; (b) hooking a tab extending generally downwardly from a portion of the floor sealant material to pull the floor sealant material off at least one drug compartment; (c) releasing dry powder from the at least one drug compartment into a target inhalation flow path; and (d) vibrating the dry powder in the flow path.

Embodiments of the present invention provide dry powder packages, inhalers, and/or methods for operating same that can be used with dry powder inhalers. In some embodiments, the drug compartments can have active piezoelectric components. Alternatively or additionally, the inhaler can have an active piezoelectric component forming a part of the flow path away from the drug compartment to facilitate fluidic drug dispersion.

It is noted that aspects of the invention may be embodied as hardware, software or combinations of same, i.e., devices and/or computer program products. These and other objects and/or aspects of the present invention are explained in detail in the specification set forth below.

### BRIEF DESCRIPTION OF THE DRAWINGS

FIG. 1A is a front perspective view of a dry powder inhaler according to embodiments of the present invention.

FIG. 1B is a front, top perspective view of a multi-dose drug package according to embodiments of the present invention.

FIG. 1C is a front, bottom perspective view of the multi-dose package shown in FIG. 1B.

FIG. 1D is a partial top (or bottom) view of another embodiment of a multi-dose package according to embodiments of the present invention.

FIG. 2A is a top perspective view of the inhaler shown in FIG. 1A, illustrated with a cover in a closed position.

FIG. 2B is a top perspective view of the inhaler shown in FIG. 2A with the cover in an open position.

FIG. 2C is a bottom perspective view of the inhaler shown in FIG. 2B.

FIG. 3 is a schematic illustration of an inhaler circuit according to embodiments of the present invention.

FIG. 4A is an exploded bottom perspective view of a portion of an inhaler illustrating the drug package shown in FIG. 1B along with a hook member according to embodiments of the present invention.

FIG. 4B is an exploded top perspective view of the components shown in FIG. 4A.

FIG. 4C is a schematic illustration of an alternate configuration of a drug package according to embodiments of the present invention.

FIGS. 4D-4F are top perspective views of alternate drug compartments according to embodiments of the present invention.

FIG. 5A is a top perspective view of the portion of the inhaler shown in FIG. 4A, illustrating an exemplary assembled relationship.

FIG. 5B is a greatly enlarged partial perspective view of the devices shown in FIG. 5A.

FIG. 6A is a greatly enlarged perspective view of the devices shown in FIGS. 5A and 5B, but illustrated with the drug compartment primary body omitted.

FIGS. 6B and 6C are schematic illustrations of a sequence of operations according to embodiments of the present invention.

FIG. 7 is an enlarged side perspective view of the flow channel and the hook member shown in FIGS. 4A and 4B.

FIG. 8 is a side perspective view of the flow channel/airpath with the drug package and hook member in position according to embodiments of the present invention.

FIG. 9 is a side cutaway view of an exemplary inhaler according to embodiments of the present invention.

FIG. 10 is a block diagram of a data processing control system according to embodiments of the present invention.

FIG. 11 is a flow chart of operations that can be used to carry out embodiments of the present invention.

### DESCRIPTION OF EMBODIMENTS OF THE INVENTION

The present invention will now be described more fully hereinafter with reference to the accompanying figures, in which embodiments of the invention are shown. This invention may, however, be embodied in many different forms and should not be construed as limited to the embodiments set forth herein. Like numbers refer to like elements throughout. In the figures, certain layers, components or features may be exaggerated for clarity, and broken lines illustrate optional features or operations unless specified otherwise. In addition, the sequence of operations (or steps) is not limited to the order presented in the figures and/or claims unless specifically indicated otherwise. In the drawings, the thickness of lines, layers, features, components and/or regions may be exaggerated for clarity and broken lines illustrate optional features or operations, unless specified otherwise.

It will be understood that when a feature, such as a layer, region or substrate, is referred to as being “on” another feature or element, it can be directly on the other feature or element or intervening features and/or elements may also be present. In contrast, when an element is referred to as being “directly on” another feature or element, there are no intervening elements present. It will also be understood that, when a feature or element is referred to as being “connected”, “attached” or “coupled” to another feature or element, it can be directly connected, attached or coupled to the other element or intervening elements may be present. In contrast, when a feature or element is referred to as being “directly connected”, “directly attached” or “directly coupled” to another element, there are no intervening elements present. Although described or shown with respect to one embodiment, the features so described or shown can apply to other embodiments.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and should not be interpreted in an idealized or overly formal sense unless expressly so defined herein.

In the description of the present invention that follows, certain terms are employed to refer to the positional relationship of certain structures relative to other structures. As used herein, the term “front” or “forward” and derivatives thereof refer to the general or primary direction that the dry powder travels as it is dispensed to a patient from a dry powder inhaler; this term is intended to be synonymous with the term “downstream,” which is often used in manufacturing or material flow environments to indicate that certain material traveling or being acted upon is farther along in that process than other material. Conversely, the terms “rearward” and “upstream” arid derivatives thereof refer to the direction opposite, respectively, the forward or downstream direction.

The term “blister” means a sealed dry powder well, compartment or receptacle that can hold a (typically meted bolus) quantity of a dry powder, typically a dry powder medicament, and is not limited to a raised surface configuration. The term “package” describes a drug container device (such as a card) that holds a plurality of sealed blisters and/or sealed compartments and may be also known as a drug containment system (“DCS”). The term “loop” means a closed or partially closed member that has a gap, aperture or space that can cooperate with a releasing member to peel, tear or otherwise remove a releasable sealant material and/or layer from a respective one or more drug well or blister. The term “sealant layer” and/or “sealant material” includes configurations that have at least one layer or one material; thus, such a phrase also includes multi-layer or multi-material sealant configurations.

The devices and methods of the present invention may be particularly suitable for holding doses of particulate dry powder substances that are formulated for in vivo inhalant dispersion (using an inhaler) to subjects, including, but not limited to, animal and, typically, human subjects. The inhalers 10 can be used for nasal and/or oral (mouth) respiratory inhalation delivery.

The dry powder substance may include one or more active pharmaceutical constituents as well as biocompatible additives that form the desired formulation or blend. As used herein, the term “dry powder” is used interchangeably with “dry powder formulation” and means the dry powder can comprise one or a plurality of constituents or ingredients with one or a plurality of (average) particulate size ranges. The term “low-density” dry powder means dry powders having a density of about 0.8 g/cm³ or less. In particular embodiments, the low-density powder may have a density of about 0.5 g/cm³ or less. The dry powder may be a dry powder with cohesive or agglomeration tendencies.

In any event, individual dispensable quantities of dry powder formulations can be a single ingredient or a plurality of ingredients, whether active or inactive. The inactive ingredients can include additives added to enhance flowability or to facilitate aeorolization delivery to the desired target. The dry powder drug formulations can include active particulate sizes that vary. The device may be particularly suitable for dry powder formulations having particulates which are in the range of between about 0.5-50 μm, typically in the range of between about 0.5 μm-20.0 μm, and more typically in the range of between about 0.5 μm-8.0 μm. The dry powder formulation can also include flow-enhancing ingredients, which typically have particulate sizes that may be larger than the active ingredient particulate sizes. In certain embodiments, the flow-enhancing ingredients can include excipients having particulate sizes on the order of about 50-100 μm. Examples of excipients include lactose and trehalose. Other types of excipients can also be employed, such as, but not limited to, sugars which are approved by the United States Food and Drug Administration (“FDA”) as cryoprotectants (e.g., mannitol) or as solubility enhancers (e.g., cyclodextrine) or other generally recognized as safe (“GRAS”) excipients.

“Active agent” or “active ingredient” as described herein includes an ingredient, agent, drug, compound, and composition of matter or mixture, which provides some pharmacologic, often beneficial, effect. This includes foods, food supplements, nutrients, drugs, vaccines, vitamins, and other beneficial agents. As used herein, the terms further include any physiologically or pharmacologically active substance that produces a localized and/or systemic effect in a patient.

The active ingredient or agent that can be delivered includes antibiotics, antiviral agents, anepileptics, analgesics, anti-inflammatory agents and bronchodilators, and may be inorganic and/or organic compounds, including, without limitation, drugs which act on the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system, and the central nervous system. Suitable agents may be selected from, for example and without limitation, polysaccharides, steroid, hypnotics and sedatives, psychic energizers, tranquilizers, anticonvulsants, muscle relaxants, anti-Parkinson agents, analgesics, anti-inflammatories, muscle contractants, antimicrobials, antimalarials, hormonal agents including contraceptives, sympathomimetics, polypeptides and/or proteins (capable of eliciting physiological effects), diuretics, lipid regulating agents, antiandrogenic agents, antiparasitics, neoplastics, antineoplastics, hypoglycemics, nutritional agents and supplements, growth supplements, fats, antienteritis agents, electrolytes, vaccines and diagnostic agents.

The active agents may be naturally occurring molecules or they may be recombinantly produced, or they may be analogs of the naturally occurring or recombinantly produced active agents with one or more amino acids added or deleted. Further, the active agent may comprise live attenuated or killed viruses suitable for use as vaccines. Where the active agent is insulin, the term “insulin” includes natural extracted human insulin, recombinantly produced human insulin, insulin extracted from bovine and/or porcine and/or other sources, recombinantly produced porcine, bovine or other suitable donor/extraction insulin and mixtures of any of the above. The insulin may be neat (that is, in its substantially purified form), but may also include excipients as commercially formulated. Also included in the term “insulin” are insulin analogs where one or more of the amino acids of the naturally occurring or recombinantly produced insulin has been deleted or added.

It is to be understood that more than one active ingredient or agent may be incorporated into the aerosolized active agent formulation and that the use of the term “agent” or “ingredient” in no way excludes the use of two or more such agents.

Examples of diseases, conditions or disorders that may be treated with embodiments of the invention include, but are not limited to, asthma, COPD (chronic obstructive pulmonary disease), viral or bacterial infections, influenza, allergies, cystic fibrosis, and other respiratory ailments as well as diabetes and other insulin resistance disorders. The dry powder inhalation may be used to deliver locally acting agents such as antimicrobials, protease inhibitors, and nucleic acids/oligionucleotides as well as systemic agents such as peptides like leuprolide and proteins such as insulin. For example, inhaler-based delivery of antimicrobial agents such as antitubercular compounds, proteins such as insulin for diabetes therapy or other insulin-resistance related disorders, peptides such as leuprolide acetate for treatment of prostate cancer and/or endometriosis and nucleic acids or ogligonucleotides for cystic fibrosis gene therapy may be performed See e.g. Wolff et al., Generation of Aerosolized Drugs, J. Aerosol. Med. pp. 89-106 (1994). See also U.S. Patent Application Publication No. 20010053761, entitled Method for Administering ASPB28-Human Insulin and U.S. Patent Application Publication No. 20010007853, entitled Method for Administering Monomeric Insulin Analogs, the contents of which are hereby incorporated by reference as if recited in full herein.

Typical dose amounts of the unitized dry powder mixture dispersed in the inhaler may vary depending on the patient size, the systemic target, and the particular drug(s). A conventional exemplary dry powder dose amount for an average adult is about 10-30 mg and for an average adolescent pediatric subject is from about 5-10 mg. A typical dose concentration may be between about 1-2%. Exemplary dry powder drugs include, but are not limited to, albuterol, fluticasone, beclamethasone, cromolyn, terbutaline, fenoterol, βagonists (including long-acting β-agonists), salmeterol, formoterol, cortico-steroids and glucocorticoids. In certain embodiments, the administered bolus or dose can be formulated with an increase in concentration (an increased percentage of active constituents) over conventional blends. Further, the dry powder formulations may be configured as a smaller administerable dose compared to the conventional 10-25 mg doses. For example, each administerable dry powder dose may be on the order of less than about 60-70% of that of conventional doses. In certain particular embodiments, using the active dispersal systems provided by certain embodiments of the DPI configurations of the instant invention, the adult dose may be reduced to under about 15 mg, such as between about 10 μg-10 mg, and more typically between about 50 μg-10 mg. The active constituent(s) concentration may be between about 5-10%. In other embodiments, active constituent concentrations can be in the range of between about 10-20%, 20-25%, or even larger. In particular embodiments, such as for nasal inhalation, target dose amounts may be between about 12-100 μg.

In certain particular embodiments, during dose dispensing, the dry powder in a particular drug compartment or blister may be formulated in high concentrations of an active pharmaceutical constituent(s) substantially without additives (such as excipients). As used herein, “substantially without additives” means that the dry powder is in a substantially pure active formulation with only minimal amounts of other non-biopharmacological active ingredients. The term “minimal amounts” means that the non-active ingredients may be present, but are present in greatly reduced amounts, relative to the active ingredient(s), such that they comprise less than about 10%, and preferably less than about 5%, of the dispensed dry powder formulation, and, in certain embodiments, the non-active ingredients are present in only trace amounts.

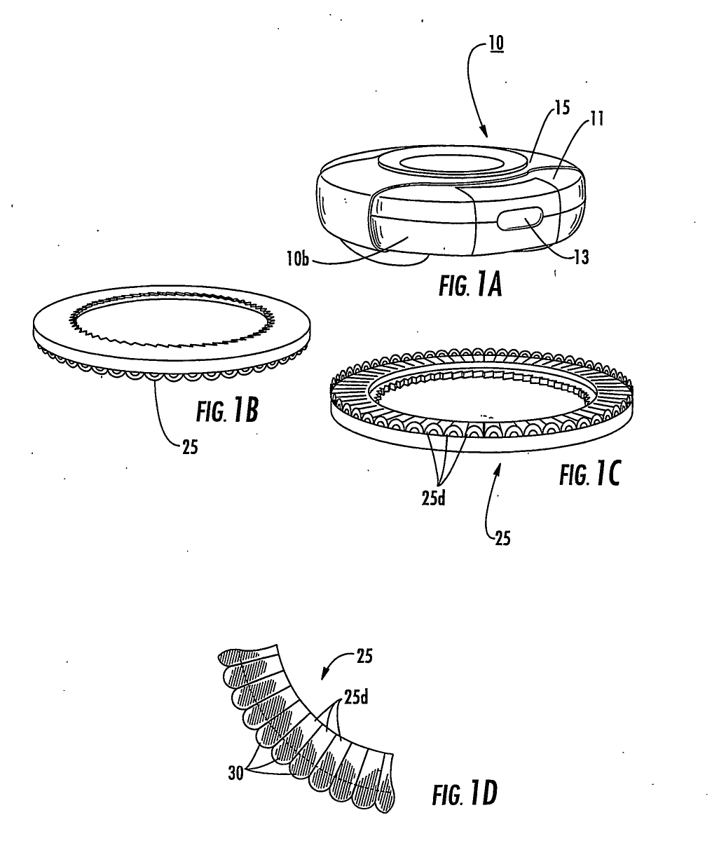

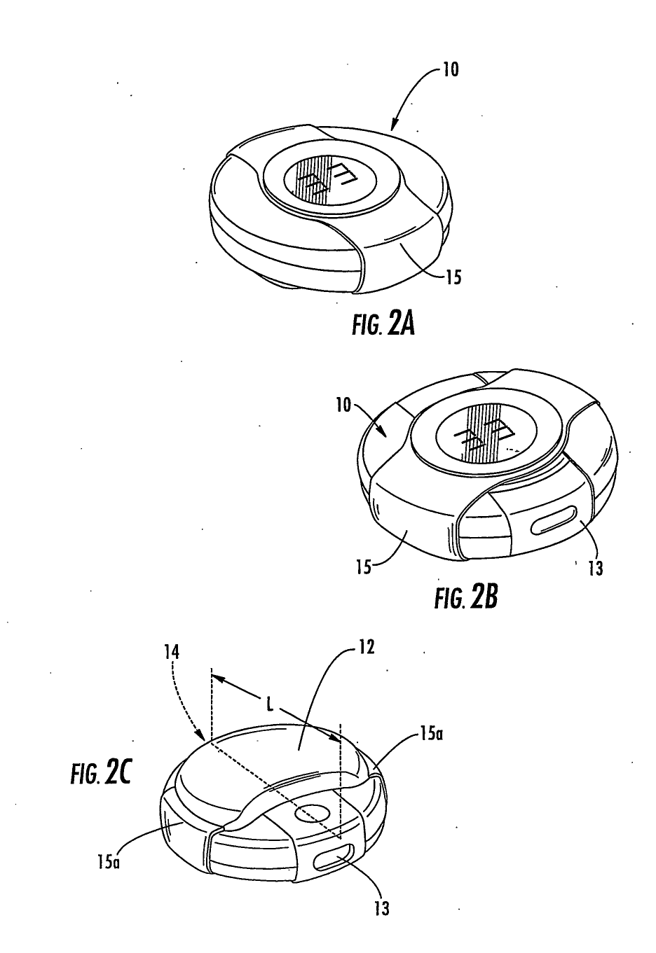

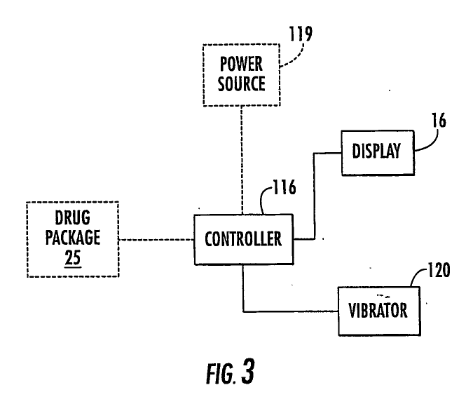

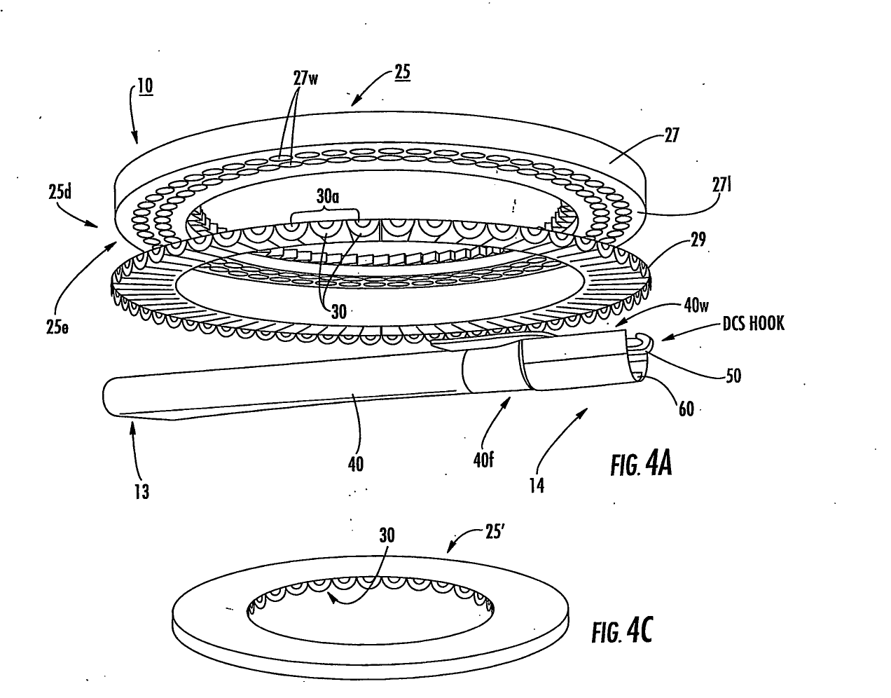

Turning now to the figures, FIG. 1A illustrates a dry powder inhaler 10 according to certain embodiments of the present invention. FIGS. 1B and 1C illustrate an example of a dry powder drug package 25 that can be configured to reside in the inhaler 10. Referring again to FIG. 1A, the inhaler 10 can have a relatively small body 10b and may include generally planar upper and/or lower primary surfaces 11, 12 (the lower primary surface 12 is shown in FIG. 2C). The inhaler 10 includes a mouthpiece port 13 and an opposing air inlet port 14. As shown in FIG. 9, the air inlet port 14 can be disposed to be generally diametrically opposed from the mouthpiece port 13, with each located in a generally common plane across at least a major portion of the length “L” of the inhaler body 10b, and with the flow path 40f typically extending across substantially the entire length of the inhaler body 10b. In some embodiments, the mouthpiece port 13 and air inlet port 14 are generally aligned and spaced apart about a distance of between about 2-5 inches, and typically between about 4-5 inches. The body 10b can have a portable relatively compact “pocket-sized” configuration that holds meted doses of dry powder in discrete spaced apart (typically at least about 30, as will be discussed further below) drug compartments. In some embodiments, the body 10b can have a width/length that is less than about 4.5 inches, typically less than about 3.5 inches, and a thickness/depth of less than about 2 inches, typically less than about 1.5 inches. The inhaler body 11 can also be configured to be generally planar on opposing primary surfaces to facilitate pocket storage.

As shown in FIGS. 2B and 2C, the inhaler 10 can also include an attachable cover 15 that can rotate to allow a user access to the mouthpiece port 14. In the embodiment shown, the cover 15 can be configured to move to concurrently expose the air inlet port 14 and the mouth port 13 during operation, then, as shown in FIGS. 1A and 2A, to rotate to encase each port 13, 14 during periods of non-use to inhibit undesired foreign objects from entering the inhaler 10. As shown in FIGS. 2B and 2C, the cover 15 can span the width of the body and include generally downwardly extending gripping arms 15a that snugly and slidably attach to an outer edge portion of a lower surface of the inhaler 10. The cover 15 is pivotally attached to rotate about a center portion of the inhaler 10. The cover 15 can comprise an elastomeric material.

As is also shown in FIGS. 1A, 2A and 2B, the inhaler 10 can include an electronic and/or digital display 16 that can be configured to display the number of doses that are available in the inhaler 10. As shown in FIG. 3, the inhaler 10 can include a controller 116 that can automatically decrement the displayed number after an active inhalation delivery and that may, in some embodiments, control the activation of a vibrator 120 in communication with the inhalation flow path to promote fluidization of the dry powder during inhalation drug delivery.

The inhaler 10 can also be configured to be able to electronically communicate with a remote location or device and/or provide additional data. The inhaler 10 can be configured with a clock and can generate patient alarms, alerts and/or reminders to take the medicine or evaluate whether a medicine is desired at target intervals or (selectable) times. The inhaler 10 can be configured to provide an “on” and/or “off” status indicator and/or generate one or more of: (a) a low battery charge warning; (b) a drug refill or expiration warning; (c) the number of available inhalant doses remaining; and/or (d) a confirmation that the drug powder was successfully delivered (the above may be provide either via a visual and/or audible signal).

The inhaler 10 can include a computer port (not shown). The port may be, for example, RS 232, infrared data association (IRDA) or universal serial bus (USB), which may be used to download or upload selected data from/to the inhaler to a computer application or remote computer, such as a clinician or other site. The inhaler 10 can be configured to communicate with a clinician or pharmacy for refills and/or patient compliance. The inhaler 10 may also include a second peripheral device communication port (now shown).

In some embodiments, the controller 116 can include computer program code and/or computer applications that communicate additional data to a user (optionally to the display) as noted above and/or communicate with another remote device (the term “remote” including communicating with devices that are local but typically not connected during normal inhalant use) device.

The inhaler 10 can house a drug delivery package 25 which can include electronic media, such as electronic memory, a microchip, or optical or other electronic indicia that can be automatically interrogated by a sensor or reader operatively associated with the controller 116 in/on the inhaler 10 and the data relayed to the controller 116 upon input into the inhaler body 10b. The drug delivery package 25 can be disposed of after use and replaced as desired. In other embodiments, the entire inhaler 10 can be disposable after dispensing its loaded drug package.

In some embodiments, the controller 116 can be programmed with a library of a plurality of desired dry powder excitation signals that can be automatically selected by the controller 116 based on the data relayed and carried by the package 25 corresponding to the drug type/drug package 25 disposed therein. In this way, customized drug signals can be used to fluidize the dry powder. In other embodiments, the dry powder excitation signal can be carried on the electronic memory (not shown) held on the drug package 25 itself and the controller 116 can be configured to output the signal on the package to a vibrator 120 operatively associated with the dry powder. Examples of suitable excitation signals are described in co-pending U.S. Patent Application Publication Nos. 2004-0025877-A1 and 2004-0123864, the contents of which are hereby incorporated by reference as if recited in full herein.

The vibrator 120 can be any suitable vibrator configuration. The vibrator 120 can be configured to vibrate the flow channel or a portion thereof In other embodiments, the vibrator 120 can also be configured to vibrate the drug compartment holding the dry powder. Examples of vibrators include, but are not limited to, one or more of: (a) ultrasound or other acoustic or sound based sources (above, below or at audible wavelengths) that can be used to instantaneously apply non-linear pressure signals onto the dry powder; (b) electrical or mechanical deflection of the sidewalls and/or floor of the inhalation flow channel and/or drug compartment; (c) solenoids, piezoelectrically active portions and the like); and (d) oscillating or pulsed gas (airstreams), which can introduce changes in one or more of volume flow, linear velocity, and/or pressure. Examples of mechanical and/or electromechanical vibratory devices are described in U.S. Pat. Nos. 5,727,607, 5,909,829 and 5,947,169, the contents of which are incorporated by reference as if recited in full herein. In some particular embodiments, the vibrator 120 includes at least one piezoelectric element, such as a piezoceramic component, and/or a piezoelectric polymer film. An example of a suitable piezoelectric vibrator 120 will be discussed further below.

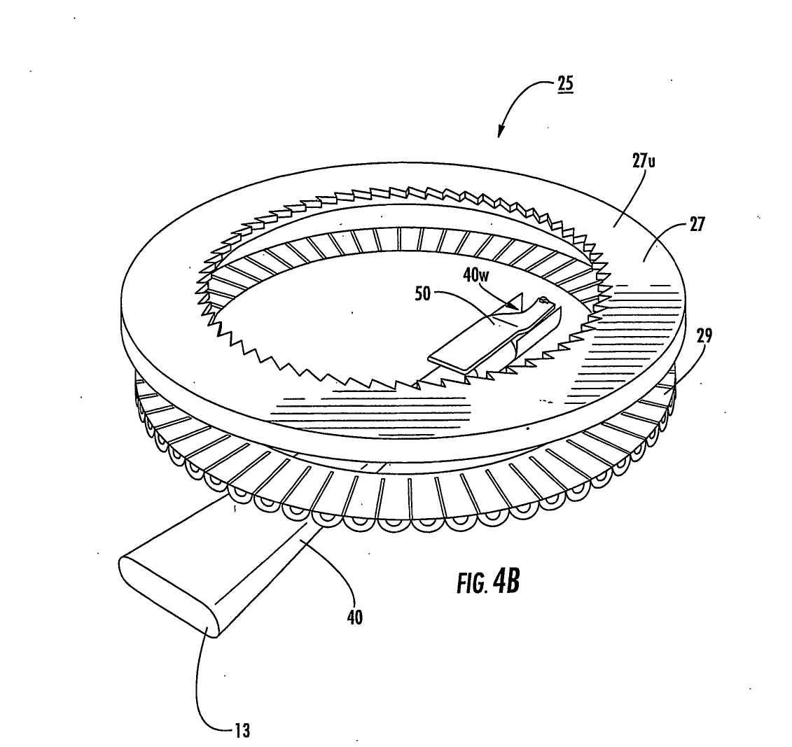

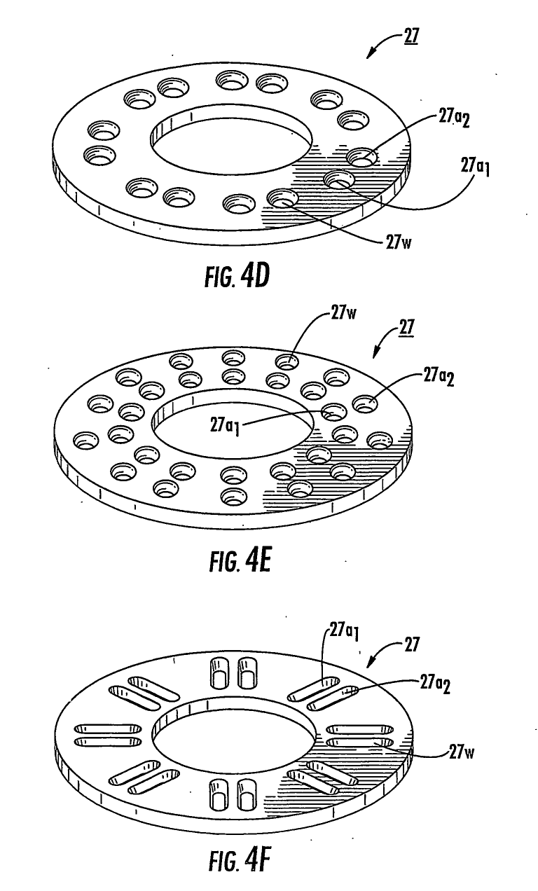

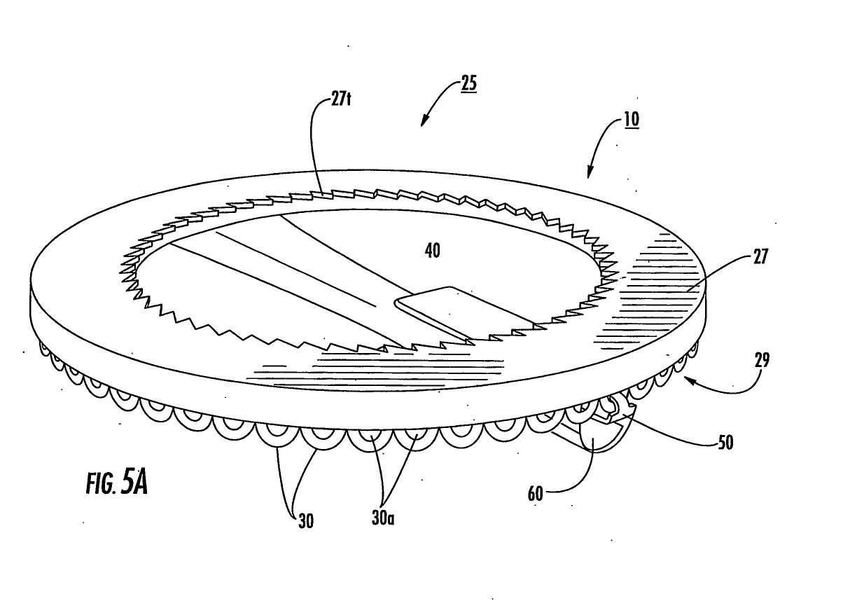

FIGS. 4A and 4B are exploded illustrations of an exemplary dry powder package 25 according to certain embodiments of the present invention. As shown in FIG. 1C, the dry powder package 25 includes a plurality of generally regularly spaced apart drug compartments 25d. The embodiment shown has a disk-like shape with circumferentially spaced apart drug compartments but other drug compartment and drug package configurations may also be used. FIG. 4A illustrates two generally concentric rows of drug compartments 25d with a pair of axially aligned drug compartments (front to back) being configured to enter the window 40w concurrently for in situ mixing and release, thereby providing combination inhalation delivery. In other embodiments, other configurations of wells 27w may be used, and/or pairs of side-by-side compartments, and/or axially and laterally offset compartments may be used.

FIGS. 4D-4F illustrate alternative configurations of the support member 27 with neighboring member apertures 27a₁, 27a₂ that define a sidewall(s) of corresponding drug compartments 25d (FIG. 4A) which can be used to concurrently dispense the dry powder in neighboring blisters for combination therapeutic treatments. As for the embodiment shown in FIG. 4A, the top surface of the support member 27 may be closed, although it is shown open in FIGS. 4D-4F. Also, the support member 27 may be configured with increased density drug compartments relative to those shown for discussion. It is contemplated that in certain embodiments, relatively shallow or small volume wells 27w will be used for certain dry powder medicaments. The neighboring apertures 27a₁, 27a₂ (which may be pairs or sets of three of more apertures) may be spaced closer together on the support member 27 than the non-neighboring apertures. Each adjacent or neighboring drug compartment may contain different medicaments and/or dry powders therein that can be concurrently released upon inhalation during operative use. FIG. 4D illustrates side-by-side, neighboring (adjacent) pairs of apertures 27a₁, 27a₂ that form the sidewall(s) of corresponding drug compartments that are configured to be jointly dispersed upon inhalation in an inhaler. FIG. 4E illustrates front to back neighboring blister sidewalls 27a₁, 27a₂ that can also configured to be jointly dispersed upon inhalation via an inhaler. FIG. 4F illustrates yet another side-by-side configuration with neighboring elongate apertures 27a₁, 27a₂. In certain embodiments, the side-by-side aperture/blister configurations may be offset lengthwise (not shown) across the blister package. See co-pending U.S. Provisional Application Ser. No. 60/514,733 for additional combination drug compartment configurations, the contents of which are hereby incorporated by reference as if recited in full herein.

In any event, in some embodiments, the drug package 25 can be configured to hold at least about 30 meted doses of dry powder in discrete spaced apart drug compartments. In some embodiments, the package 25 is configured with increased density compartments, typically at least about 60, and may be configured with between about 90-120, or more compartments. The meted contents of two or more of the compartments may be mixed together in situ during release/inhalation to provide combination drug therapies and/or mixing at a time of use.

The drug compartments 25d are closed by a detachable sealant material 29, shown as a floor in the embodiment shown in FIG. 4A. Meted and/or bolus amounts of dry powder can be disposed in the drug compartments and sealed in the drug compartment via the sealant material 29. The sealant material 29 can comprise foil, TEDLAR, or other moisture resistant barrier material. The sealant material 29 can be configured as a thin flexible and/or resilient film. For example, the sealant material 29 can contain foil (such as aluminum or other suitable material) and/or may be a laminated structure, comprising a polymer layer and a foil layer. In particular embodiments, the sealant 29 can comprise layers of polyamide film, aluminum foil, and polypropylene film (where the top layer may be the polyamide film) adhered to each other with a laminating adhesive. In certain embodiments, the sealant 29 can have a thickness of between about 0.100-0.150 mm, and typically about 0.127 mm.

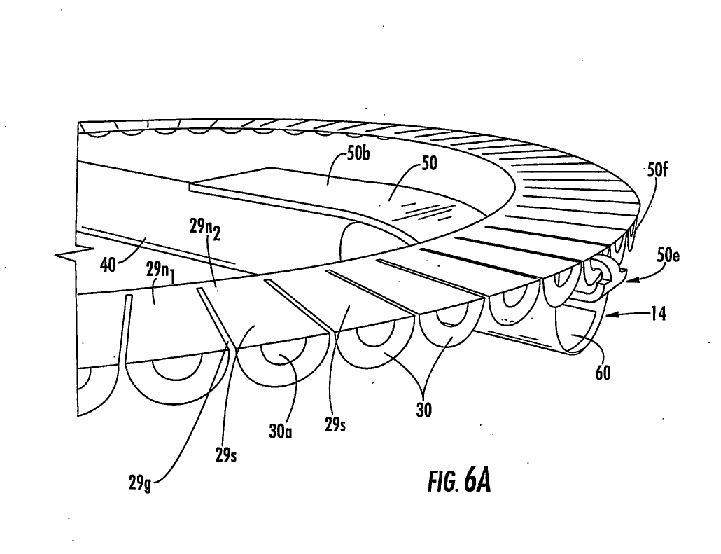

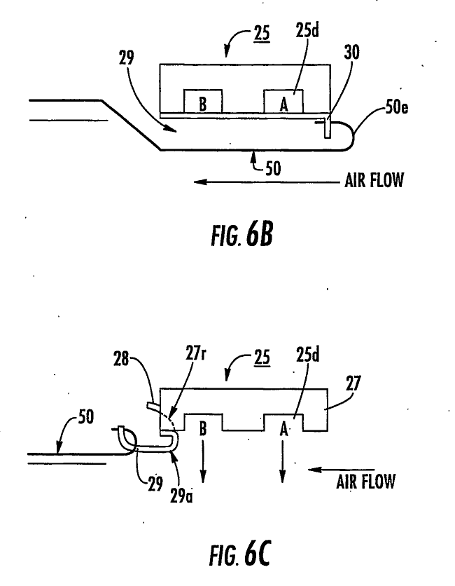

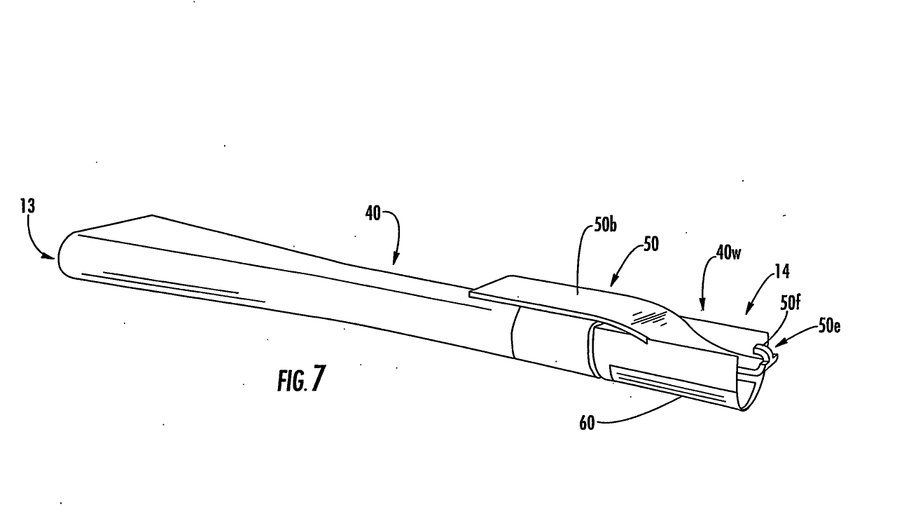

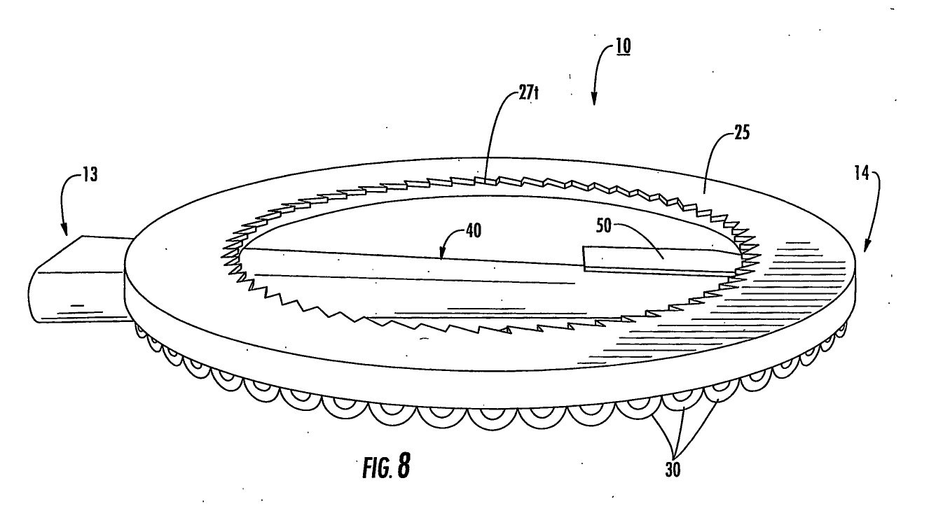

As is also shown, the drug package 25 can include a plurality of tab members 30. As is also shown in FIGS. 4A and 4B, the inhaler 10 can also include a hook member 50 that is configured to engage a selected tab member 30 and pull the sealant material 29 off of the operatively associated drug compartment(s) 25d held in the dispensing location above the dry powder window 40w. The sealant material 29 can be configured in discrete strips 29s (FIG. 6A) that cover one or more selected drug compartment 25d or may be configured as a unitary sheet that is scored or otherwise configured to preferentially release from either side of a respective drug compartment(s) 25d as the associated tab member 30 is pulled by the hook member 50 away from the body of the support member 27.

As shown in FIG. 6A, the sealant material 29 is configured as a unitary layer of strips attached at the end portion opposing the tab members 30, and includes axially extending gap spaces 29g between neighboring strips 29n₁, 29n₂. Each strip 29s can cover one drug compartment or, as shown in FIG. 4A, a plurality of drug compartments 25d, and is operatively associated with a respective tab member 30. As the portion of sealant material 29 is removed (pulled away) from a respective compartment 25d, it can remain attached to the package body 25 but held away from the associated dispensing well or cavity 25w to allow free migration of the dry powder into the window 40w of the airflow path.

The tab members 30 can extend in a generally downward direction and be attached to a portion of the sealant material 29. The tab members 30 can be configured as loops, and in some particular embodiments, as shown, the loops may be configured as a closed perimeter shape enclosing an aperture 30a. In other embodiments, the loops may be configured as open perimeter structures (not shown). The tab members 30 can be configured to include at least a portion that has increased structural rigidity relative to that of the sealant material 29. For example, the tab members 30 can include an additional layer of material, a reinforcing coating material, a thicker or supplemental increased rigidity material, or combinations of same. FIG. 1B illustrates that the tab members 30 have sufficient structural rigidity to substantially retain their shape when supporting the package 25. The tab members 30 can be adhesively attached or fused to a leading edge portion (the leading edge being defined facing the inhaler user) of the sealant material so that the tab members 30 have a peel strength with the sealant material 29 that is greater than that of the sealant material 29 to the member 27. In one embodiment, the tab member 30 can comprise TYVEK® polymer formed as a generally forward arcuate edge portion with the gap 30a that extends off the sealant layer 29. In other embodiments, the tab members 30 can be formed from an extension of the sealant material (not shown).

In some embodiments, the tab members 30 may be formed without an opening (such as shown in FIG. 1D), as the hook or sealant release member can be otherwise configured to engage the tab member in order to hold, grasp or slice through to engage a respective tab member 30 and pull the sealant from the desired well(s) 27w. For example, magnetic means, pinching means, piercing means or other attachment means can be used. The tab members 30 may have a curvilinear forward edge portion or may comprise other shapes, such as, but not limited to, strips, generally triangular shapes, and the like.

As shown, the tab members 30 can be disposed proximate an outermost edge portion 25e of the dry powder package 25. In the embodiment shown, the intact tab members 30 reside in a downstream most location of the associated drug compartment(s) 25d. In other embodiments, as shown in FIG. 4C, the tab members 30 can reside upstream of the associated drug compartment(s) 25d with the tab members positioned proximate an innermost portion of the drug package 25′.

Although shown as defining the floor of the drug compartments 25d, in other embodiments the sealant material 29 can define the ceiling with the tab members 30 alternatively oriented to extend generally upwardly. In some particular embodiments, the tab members 30 of one or more drug compartments 25d can be oriented to extend outwardly generally horizontally as shown in FIG. 1D.

In the embodiment shown in FIGS. 4A and 4B, the drug package 25 includes a support member 27 with opposing upper and lower primary surfaces 27u, 27l. The member 27 includes a plurality of spaced apart cavities or wells 27w. The support member 27 can be a unitary member that comprises a generally rigid elastomeric material. The phrase “generally rigid” means that the body of the support member 27 may flex somewhat but is structurally sufficiently rigid to maintain its shape when the other components are assembled thereto.

In particular embodiments, the support member 27 can be a substantially rigid light-weight member that provides structural integrity to the package 25. The member 27 may have a unitary body as shown or a laminated or stacked structure (not shown). In certain embodiments, the member 27 can be a molded polymer or fiber reinforced resin material. In particular embodiments, the member 27 comprises a natural homopolymer polypropylene and can typically be between about 1-3 mm thick, and is more typically about 2 mm thick. Other suitable materials or fabrication methods and thicknesses can also be used. The member 27 may include additional apertures, slots or depressions and the like to further decrease weight as suitable. The member 27 may be configured with sufficient thickness, material and/or coatings to provide a moisture barrier (inhibit moisture penetration) to the dry powder held in the blister as discussed above for the sealant.

As shown in FIG. 4B, the member 27 can be configured so that the upper primary surface 27u is generally closed and configured to define the ceilings of the drug compartments 25d. Thus, as shown, the unitary member 27 can define the sidewalls and ceiling of the drug compartments 25d. In other embodiments (such as shown in FIGS. 4D-4E), the cavities or wells 27w can extend through the upper and lower surfaces 27u, 27l1, and a film or other suitable material can define the ceiling and enclose the cavities or wells 25w. For example, a thin flexible material and/or a piezoelectric polymer film or other piezoelectric material with active portions can be used to cooperate with and/or define ceiling of the drug compartment (not shown) to thereby flex the dry powder out of a target compartment 25d when in the active release position. In some embodiments, the dry powder can be primarily gravity fed into an underlying dry powder window 40w (FIGS. 4A and 4B) in the flow channel 40 when the sealant material 29 is removed. The member 27 can have a width and/or length that is about 4.5 inches or less and the drug compartment cavities 27w can have a thickness that is about 0.25 inches or less.

As is also shown in FIGS. 4A and 4B, the inhaler 10 can include an elongate flow channel 40 (also called a flow path) that merges with the mouth port 13 on one end portion and the air inlet port 14 on the other opposing end portion. Turbulence enhancers can be positioned in the flow path to promote turbulence along the flow path, typically downstream of drug compartment/dry powder entry. For example, the flow path 40 can include one or more of tabs, fingers, and/or a flow path geometry that can promote turbulence, and may slow the powder flow as it approaches and/or exits the inhaler 10 at the mouthpiece port 12, while reducing any dry powder trapping, can be used (not shown). The flow channel 40 can include a dry powder entry window 40w that resides under the target dry powder compartment(s) 25 held in the active dispensing position. The flow channel 40 can include an elongate generally tubular member. In other embodiments, the inhaler 10 can be configured to define a generally tubular channel upon assembly. For example, the housing 11 can be molded as a unitary or multi-piece configuration to define the elongate flow channel. The vibrator 120 (FIG. 3) can be configured to vibrate the flow channel 40. An exemplary vibrator configuration will be discussed further below.

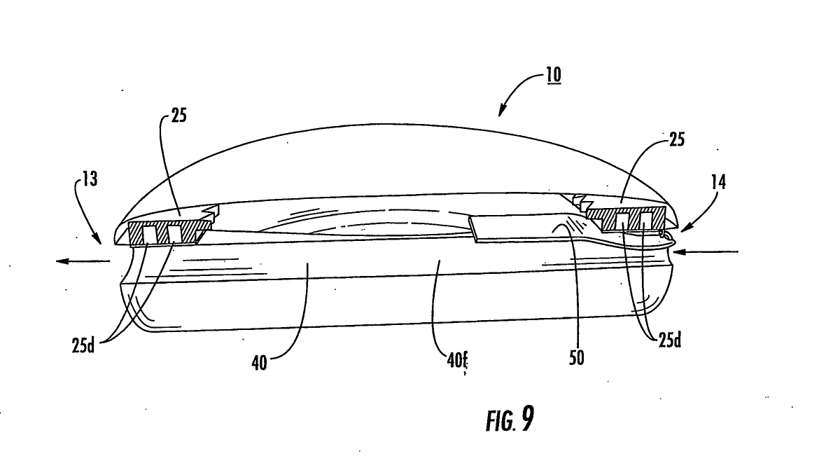

Referring to FIGS. 6A-6C and 7, as shown, the hook member 50 can be configured with a forward edge portion 50e that rises above the primary body 50b of the hook member 50. The hook member 50 can include an elongate primary body portion 50b that merges into the curvilinear secondary portion 50e that is disposed above the primary portion 50b. The secondary portion 50e can have a forward edge portion 50f that faces the direction of the mouth port 13. The hook member 50 can be configured to translate generally horizontally rearward and forward proximate and below the elongate channel window 40w.

FIGS. 6B and 6C illustrate exemplary sealant removal operations. FIG. 6B illustrates that the hook member 50 engages the tab member 30 that is operatively associated with a sealant material 29 under drug compartments A and B. FIG. 6C illustrates that the hook member 50 pulls the sealant material 29 away from (downstream) of the drug compartments 25d and a portion of the sealant material 29a can remain attached to the body 27.

As shown in FIG. 6C, the hook member 50 can be spring loaded and/or flexibly mounted in the inhaler body to flex about the vertical axis and/or generally act as a cam follower against the primary body 27 of the package 25. The hook member 50 can translate up out of the airflow path 40f as it exits the bounds of the package body 27. FIG. 9 illustrates that the hook member 50 can be configured to rise above the flow path 40f as the hook member 50 travels in a downstream direction. The support body 27 can include a ramp (shown in broken line) to help direct the hook member 50 to travel down below the drug compartment 25d and above the window 40w. The support body 27 can also optionally include a stop member 28 that, as the hook member 50 rises above the lower bounds of the support body 27, can force the hook member 50 down to deflect the hook member 50 and shake the drug compartments 25d to facilitate drug release.

Thus, generally stated, in operation, the dry powder package 25 rotates the drug compartments 25d into an active dispensing position in the inhaler 10 above the window 40w. The hook member. 50 is configured to engage a tab member 30, travel downstream toward the mouth port 13, and peel the sealant material 29 off one or more drug compartments 25d held in the active dispensing location in the inhaler 10 to release the dry powder held therein into the window of the elongate channel 40w.

It is also noted that the drug package 25 can include multiple rows of drug compartments for additional numbers (increased density) of drug doses held by the package, but the hook member 50 can be configured to open a single drug compartment 25d during a respective single delivery by limiting the travel of the hook member 50 relative to the drug package 25. Thus, for example, in this embodiment, the hook member 50 can follow a short partial stroke to pull open the strip partially to open the forwardmost drug compartment during a first inhalation delivery, then complete the stroke by pulling the strip off the second drug compartment to open the second drug compartment in the second row at a second inhalation (not shown). The drug package 25 can then be rotated to repeat the drug compartment opening order for the next set of drug compartments.

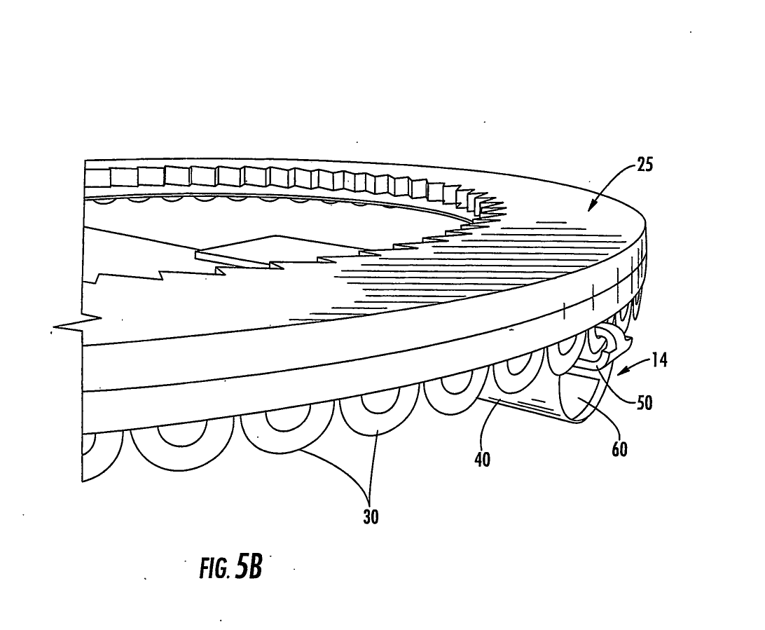

The support member 27 can also include rotating means to index a target drug compartment(s) 25d into the active dispensing position. For example, in the embodiment shown in FIGS. 5A and 8, the support member 27 includes gear teeth 27t disposed on an interior portion thereof. As is known to those of skill in the art, the inhaler 10 can include a pawl system that engages the teeth 27t and rotates the body 25 a desired incremental distance. See, e.g., U.S. Provisional Application Ser. No. 60/514,671, the contents of which are hereby incorporated by reference as if recited in full herein.

In some embodiments, the hook member 50 can be attached to a retractable mechanism that can initiate the forward and rearward movement of the hook member 50. For example, an extendable shoulder or mouthpiece can be used to push the hook member 50 forward into position, then retract the hook member 50 (move the hook member toward the user) to remove the sealant. See U.S. Provisional Patent Application Ser. No. 60/542,990, the contents of which are hereby incorporated by reference as if recited in full herein.

In some embodiments, the pivoting/rotating cover 15 (FIG. 1) can optionally turn the inhaler 10 “on” or “off” upon movement. The cover 15 can be moved into operative position manually and/or electronically. In some embodiments, the movement of the cover 15 can electronically initiate a priming or pre-vibration of at least the drug compartment 25 in an active dispensing position in the inhaler 10 before and/or during when the tab member 30 and associated covering (floor or ceiling) is removed from the drug compartment 25d (e.g., peeled from the floor in some embodiments). This vibration can shake the dry powder off the inner surfaces of the drug compartment 25d. The vibration can be carried out in any suitable manner. That is, the inhaler 10 can include a second piezoelectric member (not shown) that is in communication with the drug compartment 25d. The second member can vibrate the drug package and/or compartment. Other vibrating means can also be used as described above for the flow channel 40. In some particular embodiments, the hook member 50 can tap the drug compartment 25d or package 25, typically before and/or after the tabbed floor (or ceiling) layer is removed.

As shown in FIG. 6A, in some embodiments, the flow channel 40 can be operatively associated with a piezoelectric vibrator 120 (FIG. 3) to promote fluidic flow of the dry powder during inhalation. In some particular embodiments, as shown in FIGS. 5A, 5B and 7, a piezoelectric member 60 can be configured to reside on, above or below the floor of the chamber 40. The piezoelectric member 60 can be electrically coupled to the controller 116 (FIG. 3). In particular embodiments, the airflow path 40f has a floor with an aperture portion and a piezoelectric polymer film can be configured to extend across (cover) the aperture portion (and may extend to the sidewalls). The piezoelectric member 60 can be configured to freely flex generally orthogonally to the direction of flow in the channel 40 in response to a predetermined input signal. The input signal may be customized to the particular powder being dispensed. See, e.g., U.S. Patent Application Publication No. US-2004-0025877-A1, the contents of which are hereby incorporated by reference as if recited in full herein. The input signal can be selected based on a programmed library of signals held in memory associated with the controller 116 as noted above.

As shown in FIG. 6A, the piezoelectric member 60 can reside generally under the drug compartment(s) 25d proximate the window 40w, with an axial length of between about 0.25-2 inches, typically less than about 1 inch, and more typically about ½ inch. The member 60 can extend a distance in a downstream direction beyond the bounds of the window 40w. In other embodiments, the piezoelectric material 60 can extend at least a major portion of the channel distance, and even generally the entire length of the flow path.

The drug compartment(s) 25d can also be operatively associated with and/or include active dispersion means to promote the dry powder exiting (i.e., vibrate, shake, flex to facilitate the particulate gravity fed thru the powder entry window into flow channel). For example, as discussed above, the ceiling of the drug compartments 25d may also be operatively associated with a piezoelectric member to facilitate drug release from the compartments 25d.

In certain embodiments, the piezoelectric member 60 comprises a layer of a piezoelectric film material, typically a polymer such as PVDF (known as KYNAR® piezo film or polyvinylidene fluoride) or its copolymers such as polyvinylidene fluoride trifluoroethylene (PVDF-TrFe) or TrFe copolymer PVDF tetrafluoroethylene (PVDF-TeFE). In particular embodiments, the piezoelectric polymer material comprises a layer of a thin PVDF film. As used herein, the term “thin film” means that the piezoelectric polymer layer is configured as a structurally flexible or pliable layer that can be sized to be about 10-200 μm thick. In certain embodiments, the piezoelectric polymer layer can be sized to be less than about 100 μm thick, typically about 20-60 μm thick, and more typically about 28 μm.

The piezoelectric substrate can include a non-piezo active layer, typically comprising aluminum foil, and may, in certain embodiments, include a polymer coating or layer on the top and/or bottom thereof. The non-active layer can be at least about 20 microns thick.

A predetermined conductive material such as metal can be applied to the piezoelectric polymer. The conductive material can be a thin layer of metal or other conductive material that can be inked, stamped, printed (including screen printed), imaged (including, but not limited to, photo-resist imaging), rolled, deposited, sprayed, or otherwise applied to (one or both primary surfaces of) the piezoelectric 60. Other methods of providing the conductive pattern can also be used, including electron beam evaporation, thermal evaporation, painting, dipping, or sputtering a conductive material or metallic paint and the like or material over the selected surfaces of the piezoelectric substrate (which may comprise a PVDF layer as noted above). In particular embodiments, alternative metallic circuits, foils, surfaces, or techniques can also be employed, such as attaching a conductive Mylar layer or flex circuit over the desired portion of the outer surface of the piezoelectric substrate layer. If flex circuits are used, they may be configured or attached to the piezoelectric substrate layer so as to be substantially transparent to the structure to reduce any potential dampening interference with the substrate layer.

Typically, upper and lower surface metal trace patterns are formed on opposing sides of a piezoelectric polymer material layer but do not connect or contact each other. For example, conductive paint or ink (such as silver or gold) can be applied onto the major surfaces of the package about the elongated channels and associated metal traces such that it does not extend over the perimeter edge portions of the piezoelectric substrate layer, thereby keeping the metal trace patterns on the top and bottom surfaces separated with the piezoelectric substrate layer therebetween. This configuration forms the electrical excitation path when connected to a control system to provide the input/excitation signal for creating the electrical field that activates the deformation of the piezoelectric substrate layer during operation. Typically, one pattern is applied to one side of the piezoelectric substrate 60 and the other side may have a different conductive pattern and/or coverage.

In some embodiments, the ceiling and/or floor of the drug compartment 25d can include a piezoelectric member (which comprise a PVDF film) that alternately or additionally vibrates the drug compartment in lieu of or in addition to the flow path 40. Thus, in some alternate embodiments, the inhaler 10 is configured with multiple actuators (each drug compartment having respective piezoelectric members formed on the drug package 25) as well as in the flow path 40 or only on the drug package 25 itself. For the latter, in operation, the drug package 25 may be configured to vibrate and the tab members 30 cooperating with the hook member 50 configured to open a target drug compartment 25d. In some particular embodiments, the drug package 25 can be mounted in a lower portion of the flow path 40 so that compartments 25d face up with the ceiling in communication with the tab members 30, and the floor of each compartment 25d include a piezoelectric member 60 (such as the PVDF film) or the floor of the package be configured so that the drug compartments share one or more piezoelectric members 60 (not shown).

The controller 116 (FIG. 3) can communicate with an excitation circuit (signal generating circuitry) configuration that is connected to the piezoelectric member 60 so that one surface operates with a positive polarity while the other surface has a negative polarity or ground, or vice versa (thereby providing the electric field/ voltage differential to excite the piezoelectric substrate). Of course, the polarities can also be rapidly reversed during application of the excitation signal (such as + to −, or +to −) depending on the type of excitation signal used, thereby flexing the piezoelectric material in the region of the receptacle portion. For a more complete discussion of the active excitation path or configuration, see U.S. application Ser. No. 10/204,609, incorporated by reference herein.

In certain embodiments, the package 25 can include visible indicia and/or can be configured to engage an inhaler to provide audible alerts to warn a user that he/she is approaching the last of the filled inhalant doses on the package 25 and/or to indicate that the dose was properly (and/or improperly) inhaled or released from the inhaler device. For example, certain dry powder dose sizes are formulated so that it can be difficult for a user to know whether they have inhaled the medicament (typically the dose is aerosolized and enters the body with little or no taste and/or tactile feel for confirmation). Thus, a sensor can be positioned in communication with the flow path 40 in an inhaler and configured to be in communication with a digital signal processor or microcontroller 116 (FIG. 3), each held in or on the inhaler. In operation, the sensor is configured to detect a selected parameter, such as a difference in weight, a density in the exiting aerosol formulation, and the like, to confirm that the dose was released.

In certain embodiments, the package 25 can include color-enhanced markings for the last few (such as the last 5) doses. The color-enhanced markings may change from darker (orange to salmon or red) or to completely different colors as the last dose or last few doses approach. Alternatively (or additionally), the multi-dose disposable package 25 may be configured with audible alert features that activate a digital signal processor or micro-controller (not shown) housed in the inhaler to generate a stored audible verbal message or warning (such as “warning, refill needed, only five doses remain”) when a desired number of doses have been administered.

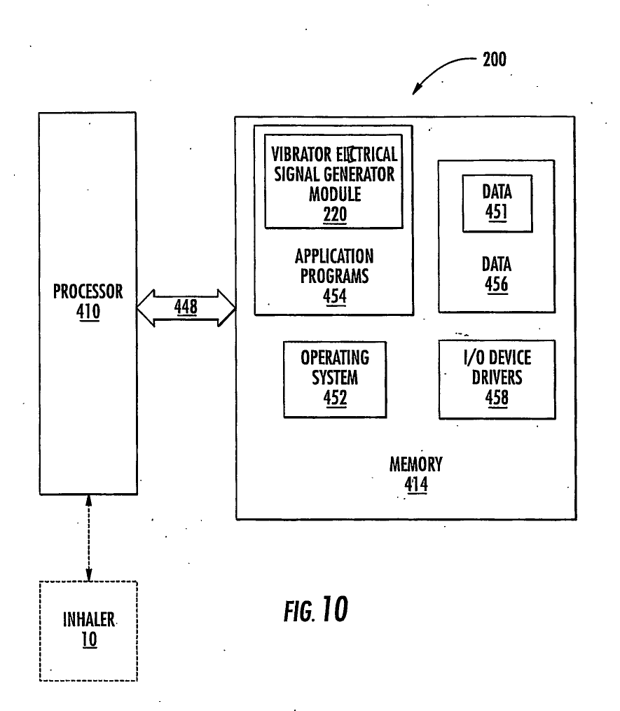

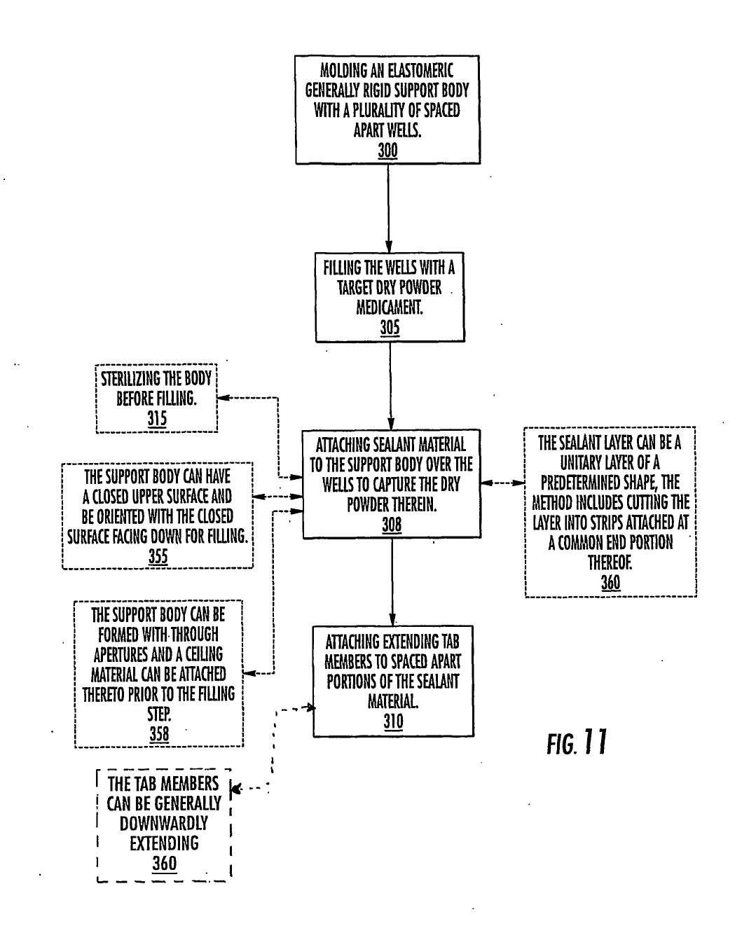

FIG. 10 illustrates an example of a control system 200 that can communicate with a signal generator circuit in the inhaler 10 and communicate with the dry powder package 25 and/or piezoelectric member 60 in the flow path 40. The control system can include a processor (such as a digital signal processor) 410 and electronic memory 414. The electronic memory can include, but is not limited to, cache, ROM, PROM, EPROM, EEPROM, flash memory, SRAM, and DRAM.

The system 200 may, in certain embodiments, also include a powder specific non-linear signal generator computer program module 220 that provides the electrical signal characteristics for the drug being dispensed. The signal generator may include a library of a priori signals for different drugs, the appropriate one of which can be selected for operation by the inhaler depending on the drug(s)in the package. The module 220 may be programmed into the memory 410. The system 200 may have a sleep or inactive (or off) mode that is turned to an active mode based on inhaler activation via input from a switch or a sensor. For example, the control system 200 may communicate with a power source 119 (FIG. 3) such as a battery (typically a miniaturized battery, such as a digital camera or pancake type flat battery) to power the signal generator and transmit the electrical signal to the piezoelectric member 60 or other vibrator means 120. The activation may be carried out automatically based upon input from a sensor and/or activation from an “on” switch.

Examples of an amplitude-modified vibratory signal suitable for vibrating the piezoelectric member 60 and/or channel 40 holding the dry powder are described in co-pending U.S. patent application Ser. No. 10/434,009, the contents of which are incorporated by reference as if recited in full herein. The vibratory signal can include a kHz carrier frequency (such as about 5 kHz-50 kHz) modified by low modulating frequency (typically about 10-200 Hz). The frequency of the vibration can be modified to match or correspond to the flow characteristics of the dry powder substance held in the package to attempt to reach a resonant frequency(s) to promote uniform drug dispersion into the body. In some embodiments, a non-linear powder-specific dry powder vibratory energy signal comprises a plurality of selected frequencies that can be generated (corresponding to the particular dry powder(s) being currently dispensed) to output the particular signal corresponding to the dry powder(s) then being dispensed. As used herein, the term “non-linear” means that the vibratory action or signal applied to the package to deliver a dose of dry powder to a user has an irregular shape or cycle, typically employing multiple superimposed frequencies, and/or a vibratory frequency line shape that has varying amplitudes (peaks) and peak widths over typical standard intervals (per second, minute, etc.) over time. In contrast to conventional systems, the non-linear vibratory signal input can operate without a fixed single or steady state repeating amplitude at a fixed frequency or cycle. This non-linear vibratory input can be applied to the blister to generate a variable amplitude motion (in either a one, two and/or three-dimensional vibratory motion). The non-linear signal fluidizes the powder in such a way that a powder “flow resonance” is generated allowing active flowable dispensing.

In particular embodiments, the package 25 can include signal-generating circuitry and/or components held thereon or therein which, in operation, are in communication with the ceilings of the drug compartments to facilitate a complete release of particulate from the drug compartment. The signal generating circuitry may be programmed with a plurality of predetermined different input signals, or if the blister package dispenses only a single dry powder, the signal generator may be programmed with a single signal. A different signal may be used to excite the piezoelectric member 60 and the ceiling of the drug compartment. Appropriate powder-specific signals, typically used for the channel vibration, can be determined experimentally and/or computationally at an OEM or evaluation site and input into the inhalers (via hardware and/or software components including programmable processors). For additional description of signals and operations to determine same, see co-pending and co-assigned U.S. patent application Ser. Nos. 10/434,009, 10/606,678, 10/607,389, and 10/606,676; the contents of these applications are hereby incorporated by reference in their entireties as if recited in full herein.

In some embodiments, a signal of combined frequencies can be generated to provide a non-linear signal to improve fluidic flow performance. Selected frequencies can be superimposed to generate a single superposition signal (that may also include weighted amplitudes for certain of the selected frequencies or adjustments of relative amplitudes according to the observed frequency distribution). Thus, the vibratory signal can be a derived non-linear oscillatory or vibratory energy signal used to dispense a particular dry powder. In certain embodiments, the output signal used to activate the piezoelectric blister channel may include a plurality of (typically at least three) superpositioned modulating frequencies and a selected carrier frequency. The modulating frequencies can be in the range noted herein (typically between about 10-500 Hz), and, in certain embodiments may include at least three, and typically about four, superpositioned modulating frequencies in the range of between about 10-100 Hz, and more typically, four superpositioned modulating frequencies in the range of between about 10-15 Hz.

While the present invention is illustrated, for example, with reference to the module 220 being an application program in FIG. 10, as will be appreciated by those of skill in the art, other configurations may also be utilized while still benefiting from the teachings of the present invention. Thus, the present invention should not be construed as limited to the configuration of FIG. 10, which is intended to encompass any configuration capable of carrying out the operations described herein.

The I/O data port can be used to transfer information between the data processing system 200 and the inhaler dispensing system controlled by the processor. These components may be conventional components such as those used in many conventional data processing systems which may be configured in accordance with the present invention to operate as described herein.

While the present invention is illustrated, for example, with reference to particular divisions of programs, functions and memories, the present invention should not be construed as limited to such logical divisions. Thus, the present invention should not be construed as limited to the configuration of FIG. 10 but is intended to encompass any configuration capable of carrying out the operations described herein.

FIG. 11 illustrates an example of operations that can be carried out to fabricate a package 25 such as that shown in FIGS. 1B and 1C according to embodiments of the present invention. An elastomeric generally rigid support body can be molded with a plurality of spaced apart wells 27w (block 300). The wells can be filled with a predetermined meted (typically bolus amount when either dispensed alone or with another meted dry powder medicament) amount of dry powder (block 305). The term “filled” includes providing an amount that is less than the volumetric capacity of the well. The support body may be sterilized prior to (after the wells are formed) depositing the dry powder therein (block 315). Sealant material is applied to form the floor of each well (block 308). Generally downwardly and/or generally upwardly extending tab members are attached to a portion of the sealant material (block 310).

The support body can be molded with an enclosed ceiling and the body oriented with the ceiling turned upside down for filling (block 355). Alternatively, the body can be molded with through apertures defining the wells and a ceiling comprising a sealant material, and, optionally, piezoelectric polymer material can be sealed to the support member with the dry powder captured therein (block 358). In some embodiments, the sealant layer is a unitary layer of material in a predetermined shape and the method can include separating the unitary sealant material into a plurality of (circumferentially spaced apart radially extending) strips attached at a common end portion (block 360).

Certain operations may be automated and/or carried out using computer programs and automated equipment.

The flowcharts and block diagrams of certain of the figures herein illustrate the architecture, functionality, and operation of possible implementations of dry powder-specific dispensing and/or vibratory energy excitation means according to the present invention. In this regard, each block in the flow charts or block diagrams represents a module, segment, or portion of code, which comprises one or more executable instructions for implementing the specified logical function(s). It should also be noted that in some alternative implementations, the functions noted in the blocks may occur out of the order noted in the figures. For example, two blocks shown in succession may in fact be executed substantially concurrently or the blocks may sometimes be executed in the reverse order, depending upon the functionality involved.

In certain embodiments, the powder specific vibration energy signals are non-linear and the inhaler can include computer program code that automatically selectively adjusts the output of the vibration energy signal based on the identified dry powder being dispensed. The vibration energy output signals for the dry powders being dispensed can be based on data obtained from a fractal mass flow analysis or other suitable analysis of the dry powder being administered to the user. The inhaler may be particularly suited to dispense low-density dry powder.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. In the claims, means-plus-function clauses, where used, are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. A multi-dose drug containment system package adapted for use in an inhaler, comprising:
a support member comprising a plurality of spaced apart drug compartments, each drug compartment having a sealant material detachably sealed thereto; and
a plurality of spaced apart tab members, a respective tab member attached to a portion of sealant material that extends over one or more drug compartments, wherein a respective tab member is operatively associated with at least one drug compartment so that, in operation, the respective tab member is configured to be grasped, such grasping causing the associated sealant material to pull away from at least one drug compartment to release a drug held therein.

2. A drug containment system according to claim 1, further comprising a meted dose of dry powder disposed in each drug compartment.

3. A drug containment system according to claim 1, wherein the tab members comprise loop members disposed proximate an outermost edge portion of the support member.

4. A drug containment system according to claim 3, wherein the support member is generally shaped as a card and/or disc and has a generally rigid elastomeric body.

5. A drug containment system according to claim 1, wherein the tab member has increased structural rigidity relative to the sealant material.

6. A drug containment system according to claim 3, wherein the loop member is configured with a closed perimeter about an aperture.

7. A drug containment system according to claim 1, wherein the tab member comprises an elastomeric material.

8. A drug containment system according to claim 3, wherein the loop members extend generally downwardly in position in an inhaler.

9. A drug containment system according to claim 7, wherein the sealant material comprises foil, and wherein the tab member has increased structural rigidity relative to the sealant material and less structural rigidity than the support member.

10. A drug containment system according to claim 1, wherein a respective tab member is operatively associated with a plurality of drug compartments.

11. A drug containment system according to claim 10, wherein each tab member is operatively associated with a first and second drug compartment, each drug compartment holding a different meted drug.

12. A drug containment system according to claim 1, wherein the support member is a unitary generally rigid elastomeric body with opposing upper and lower primary surfaces and a plurality of cavities formed therein, the cavities being open at the lower primary surface and defining the drug compartments.

13. A drug containment system according to claim 12, wherein the upper primary surface of the support member defines a closed generally planar ceiling over all of the cavities.

14. A drug containment system according to claim 13, wherein the sealant material of each drug compartment is attached to the lower primary surface of the support member, and wherein the tab members are disposed at an outermost edge portion of the lower primary surface of the support member and extend away from the lower primary surface thereof in a generally downward direction in position in an inhaler.

15. A drug containment system according to claim 14, wherein the sealant material is a unitary layer that extends across the lower primary surface of the support member and includes preferentially weakened release regions in communication with each tab member, the regions spanning selected neighboring pairs of drug compartments.

16. A drug containment system according to claim 14, wherein the sealant material is configured as a plurality of sealant material strips, one of each of which is associated with a respective tab member.

17. A drug containment system according to claim 16, wherein each strip extends across a plurality of neighboring spaced apart drug compartments and, in operation, is configured to expose the neighboring plurality of drug compartments for generally concurrent release.

18. A drug containment system according to claim 1, wherein each tab member is operatively associated with at least two neighboring drug compartments, and wherein the neighboring drug compartments each hold a meted amount of a different dry powder drug to thereby allow combination inhalation dry powder drug delivery in operative position in an inhaler.

19. A drug containment system according to claim 1, wherein the plurality of drug compartments is at least 60.

20. A drug containment system according to claim 19, wherein the plurality of drug compartments is between about 90 to about 120.

21. A drug containment system according to claim 3, wherein the loop members extend generally horizontally in operative position in an inhaler.

22. A drug containment system according to claim 19, wherein the support member has a generally circular profile, and wherein the plurality of drug compartments comprise a first plurality of circumferentially spaced apart drug compartments disposed about a substantially common first radius extending from a center of the support member and a second plurality of circumferentially spaced apart drug compartments disposed about a second substantially common longer radius extending from the center.

23. A drug containment system according to claim 22, wherein each tab member is operatively associated with one drug compartment disposed in the first radius location and one second drug compartment disposed in the second radius location.

24. A drug containment system according to claim 19, wherein the support member has a width and length that is about 4.5 inches or less.

25. A drug containment system according to claim 12, wherein the support member drug compartment cavities have a thickness that is less than about 0.25 inches, and wherein the drug containment system is disposable after the drug or drugs therein have been dispensed in an inhaler.

26. A dry powder inhaler comprising:
an elongate chamber having opposing first and second end portions, a floor, and a ceiling with dry powder entry window, the first end portion merging into an inhaler mouth port and the second end portion merging into an air inlet port such that the mouth and air inlet ports are in fluid communication;
a vibrator operatively associated with a portion of the elongate chamber; and
a multi-dose dry powder package comprising a plurality of spaced apart discrete meted amounts of particulate dry powder in respective sealed drug compartments, wherein, in operation, at least one of the compartments is configured to align with the chamber entry window to release at least one meted amount of dry powder therein.

27. A dry powder inhaler according to claim 26, wherein the vibrator is configured to contact the elongate chamber floor, and wherein the inhaler port and the air inlet port are generally axially aligned about opposing end portions of the inhaler and configured to be oriented about a generally horizontal plane during inhalation.

28. A dry powder inhaler according to claim 26, wherein the dry powder inhaler has a length from a forward edge portion to a rearward edge portion thereof when held in an operative position, and wherein the elongate chamber has a length that extends across at least a major portion of the length of the inhaler.

29. A dry powder inhaler according to claim 26, wherein the elongate chamber has generally the same cross-sectional area for greater than a major portion of the length of the inhaler.

30. A dry powder inhaler according to claim 28, wherein the elongate chamber is generally tubular along at least a major portion of its length.

31. A dry powder inhaler according to claim 28, wherein the elongate chamber runs substantially the entire length of the inhaler with the air inlet port located on an outermost edge portion of the inhaler and the mouth port located on a diametrically opposed outermost edge portion of the inhaler.

32. A dry powder inhaler according to claim 26, wherein the chamber window is disposed closer to the air inlet port than the mouth port.

33. A dry powder inhaler according to claim 26, wherein the dry powder package is generally configured as a disk, wherein each drug compartment comprises a releaseable floor, and wherein, in operation, the dry powder package is configured to cooperate with the inhaler to rotate and orient at least one drug compartment to overlie the elongate channel window for active dispensing.

34. A dry powder inhaler according to claim 33, wherein the dry powder package has a generally structurally rigid unitary primary body with cavities defining a ceiling and sidewalls of respective drug compartments, and wherein the floors comprise a flexible material and are configured to extend across the respective cavities to define a generally planar sealant layer.

35. A dry powder inhaler according to claim 26, further comprising a display configured to display the number of doses available in the inhaler.

36. A dry powder inhaler according to claim 35, wherein the dry powder package is disposable after the meted amounts of dry powder are used, the inhaler further comprising a controller in communication with the display, the controller configured to communicate with the disposable multi-dose dry powder package held therein, and wherein the controller is configured to automatically determine the number of doses available for dispensing on the disposable dry powder package and automatically count down and display the number available.

37. A dry powder inhaler according to claim 26, wherein the drug compartments comprise a detachable sealant material, the inhaler further comprising a translating hook member held in the inhaler, wherein, in operation, the hook member is operatively associated with one or more drug compartments as the dry powder package rotates the drug compartments into an active dispensing position in the inhaler, the hook member configured to engage and peel the sealant material off the one or more drug compartments held in the active dispensing location and release the dry powder held therein into the window of the elongate channel.

38. A dry powder inhaler according to claim 37, wherein, in operative position, the hook member is held generally horizontally below at least one dry powder package compartment proximate the elongate channel window.

39. A dry powder package according to claim 38, wherein the hook member is configured with an elongate primary portion that merges into a curvilinear secondary portion that is disposed above the primary portion, the secondary portion having a forward edge portion thereof that faces the direction of the mouth port.

40. A dry powder inhaler according to claim 37, wherein, in operation, the hook member translates in the inhaler toward the mouth port to pull the detachable sealant material off one or more target drug powder compartments.

41. A dry powder inhaler according to claim 37, wherein, after removal from a respective drug compartment by the hook member, the sealant material remains attached to the dry powder package such that the respective at least one drug compartment has a lowermost portion that is open to allow the dry powder to fall through the window unimpeded by the sealant material.

42. A dry powder inhaler according to claim 37, wherein the hook member curvilinear portion is disposed closer to the air inlet port than the mouth port.

43. A dry powder inhaler according to claim 26, wherein each drug compartment comprises a floor comprising a detachable sealant material, and wherein the dry powder package further comprises a plurality of generally downwardly extending tab members attached to selected portions of the floor sealant material proximate one or more drug compartments.

44. A dry powder inhaler according to claim 43, wherein the tab members are circumferentially spaced apart and attached about an outermost edge portion of the dry powder package.

45. A dry powder inhaler according to claim 43, wherein the tab members are configured as loop members.

46. A dry powder inhaler according to claim 43, wherein the tab members are configured with a closed loop perimeter surrounding an aperture.

47. A dry powder inhaler according to claim 26, further comprising a moveable mouthpiece cover that is rotatable to expose the mouth port and the air inlet port.

48. A dry powder inhaler according to claim 47, wherein the mouthpiece cover is pivotally attached to the inhaler and is configured to rotate to concurrently expose both the mouth port and the air inlet port during operative use, then rotate to concurrently cover both the mouth port and the air inlet port during non-use.

49. A dry powder inhaler according to claim 26, wherein the vibrator comprises a piezoelectric member.

50. A dry powder inhaler according to claim 49, wherein the elongate chamber comprises a piezoelectric polymer film in communication with a floor portion thereof, the piezoelectric polymer film being generally aligned across from the channel dry powder entry window, the piezoelectric polymer film defining a portion of the vibrator.

51. A multi-dose dry powder inhaler, comprising:
an inhaler having a housing body with a mouthpiece port and a spaced apart air inlet port disposed upstream thereof;
a multi-dose dry powder package held in the inhaler, the package comprising a plurality of spaced apart drug compartments with a meted amount of dry powder drug held therein, each compartment operatively associated with a tab member, held in the inhaler; and
a hook member disposed in the inhaler to translate between forward and rearward positions to engage a target tab member and pull thereon, thereby selectively pulling the associated floor sealant material off of at least one drug compartment during operation.

52. An inhaler according to claim 51, wherein the dry powder package comprises:
a unitary dry powder package body comprising opposing top and bottom primary surfaces with a plurality of spaced apart wells having a depth formed therein, a respective well defining at least a portion of a respective drug compartment; and
a detachable floor sealant material extending across each drug compartment and sealably attached to the bottom primary surface of the dry powder package body to capture the dry powder in a respective drug compartment, wherein the tabs are configured as generally downwardly extending spaced apart tabs attached to an outermost edge portion of a portion of the floor sealant material proximate at least one drug compartment.

53. An inhaler according to claim 51, further comprising an elongate inhalation drug flow path chamber and a vibrator disposed in the inhaler housing body in communication with the inhalation drug flow path chamber.

54. An inhaler according to claim 52, wherein the unitary body is a generally rigid elastomeric material and is configured with a generally closed upper surface that defines a ceiling and at least a portion of a sidewall of a respective drug compartment.

55. An inhaler according to claim 51, wherein each tab member is operatively associated with a neighboring first and second drug compartment, and wherein the neighboring first and second drug compartments comprise a different meted drug.

56. An inhaler according to claim 52, wherein the drug compartments are configured as pairs of drug compartments, each pair of drug compartments holding a different meted dry powder drug therein, a common sealant material segment defining the floor of each pair of compartments, the common sealant material segment operatively associated with a single tab, wherein, in operation, the hook member engages a single tab and the common sealant material segment is pulled off of the pair of compartments to release both drugs therein for generally concurrent release into the inhaler to thereby allow combination inhalation dry powder drug delivery in operative position in an inhaler.

57. An inhaler according to claim 51, wherein the plurality of drug compartments is at least 60.

58. An inhaler according to claim 51, wherein the plurality of drug compartments is at least 90.

59. An inhaler according to claim 51, wherein the plurality of drug compartments is between about 90 to about 120.

60. An inhaler according to claim 52, wherein the dry powder package body has a generally circular perimeter when viewed from the top or bottom, and wherein the plurality of drug compartments comprise a first plurality of circumferentially spaced apart drug compartments disposed about a substantially common first radius extending from a center of the package and a second plurality of circumferentially spaced apart drug compartments disposed about a second substantially common longer radius extending from the center, with pairs of drug compartments defined by one from the first radius and one from the second radius.

61. An inhaler according to claim 60, wherein each pair of drug compartments are operatively associated with a common single tab.

62. An inhaler according to claim 61, wherein the tab is a loop member.

63. An inhaler according to claim 62, wherein the tab has a closed perimeter surrounding an aperture, and wherein, in operation, the hook member is configured to extend through the aperture and capture a lower portion of the tab.

64. An inhaler according to claim 63, wherein the tab comprises an elastomeric material that has increased structural rigidity relative to that of the floor sealant material.

65. A method of operating an inhaler, comprising;
moving at least one drug compartment held on a dry powder package into a dispensing position above a dry powder entry window in an inhaler, the dry powder package having a plurality of sealed drug compartments, each having a meted amount of dry powder held captured therein above a releaseably attached floor sealant material;
engaging a tab extending generally downwardly from a portion of the floor sealant material;
pulling the tab to concurrently pull the floor sealant material off of at least one drug compartment;
releasing dry powder from the at least one drug compartment into a target inhalation flow path; and
vibrating the dry powder in the flow path.

66. A method according to claim 65, wherein the engaging step comprises extending a hook member into a generally downwardly extending loop portion of a respective tab and pulling the associated floor sealant material in a generally forward direction toward a user.

67. A method according to claim 65, wherein the moving step comprises moving a plurality of target drug compartments into the dispensing position, and wherein the engaging step engages a single tab and removes floor sealant material from the plurality of drug compartments to allow in situ mixing of different dry powder drugs for drug combination delivery.

68. A method according to claim 65, further comprising rotating a cover member attached to the inhaler to concurrently expose a mouth port and an air inlet port disposed upstream thereof, the mouth port and the air inlet port being disposed in generally diametrically from each other proximate outermost edge portions of the inhaler.

69. A method according to claim 68, wherein the tabs include a loop with an aperture that extends generally downwardly and is held proximate an outermost perimeter portion of the dry powder package, and wherein the engaging step comprises generally horizontally translating a hook so that a portion thereof enters the aperture and captures a portion of the loop to thereby pull the floor sealant material off the associated at least one drug compartment.

70. A method according to claim 65, wherein the window is disposed proximate an outermost portion of the inhaler proximate the air inlet path, and wherein the target flow path extends across an elongate flow channel that has a length that is generally coextensive as a length of the inhaler.

71. A method according to claim 65, further comprising displaying to a user a number associated with doses available for inhalation.

72. A method according to claim 71, further comprising automatically decrementing the number proximate in time to inhalation.

73. A method according to claim 65, wherein the vibrating step comprises flexing a piezoelectric polymer film disposed in the inhalation flow path to vibrate the dry powder.

74. A method according to claim 65, further comprising extending and retracting a mouthpiece on the inhaler to initiate and/or carry out the engaging step.
